SEQUENCE LISTING (4 sequences)

SEQ ID NO: 1 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - protein, 76 aa, Homo sapiens
HPLGSPGSASDLETSGLQEQRNHLQGKLSELQVEQTSLEPLQESPRPTGVWKSREVATEGIRGHRKMVLYTLRAPR

SEQ ID NO: 4 - protein, 32 aa, Homo sapiens
SPKMVQGSGCFGRKMDRISSSSGLGCKVLRRH

(19) United States
(12) Patent Application Publication
(10) Pub. No.: US 2025/0076318 A1
(43) Pub. Date: Mar. 6, 2025

(54) DEVICES AND METHODS FOR THE DETECTION OF proBNP

(71) Applicant: Analog Devices, Inc., Wilmington, MA (US)

(72) Inventors: Supriya BALAJI RAMACHANDRAN, Wilmington, MA (US); Joyce WU, Somerville, MA (US); Christophe ANTOINE, Lexington, MA (US)

(21) Appl. No.: 18/725,585

(22) PCT Filed: Dec. 29, 2022
(86) PCT No.: PCT/US2022/082510

§ 371 Date: Jun. 28, 2024

Related U.S. Application Data
(60) Provisional application No. 63/295,091, filed on Dec. 30, 2021.

(51) Int. Cl. G01N 33/74; G01N 33/543

(52) U.S. Cl. CPC G01N 33/74; G01N 33/54386; G01N 2333/58

(57) ABSTRACT

The present disclosure provides a device for determining the presence of BNP, proBNP and NT-proBNP. The devices comprise multiple capture regions configured to selectively capture BNP, proBNP and NT-proBNP by specific binding of proBNP. Alternatively or additionally, the devices may capture and label epitopes to distinguish proBNP from NT-proBNP or BNP.

### CROSS REFERENCE TO RELATED APPLICATIONS

This application is a 35 U.S.C. § 371 National Stage Application of International Patent Application No. PCT/US2022/082510, filed Dec. 29, 2022, which claims the benefit of and priority to U.S. Provisional Patent Application No. 63/295,091, filed Dec. 30, 2021, the contents of which applications are hereby incorporated by reference herein in their entireties.

### SEQUENCE LISTING

The present specification makes reference to a Sequence Listing, which was submitted electronically as an .xml file named “2022-12-21_APD7695PCT01-P49798WO_Sequence Listing” on Dec. 29, 2022. The .xml file was generated on Dec. 21, 2022 and is 5 KB in size. The entire contents of the sequence listing are hereby incorporated herein by reference.

### FIELD OF THE DISCLOSURE

This disclosure relates to devices and methods for the detection of proBNP, in particular devices and methods for the detection of proBNP, BNP, and NT-proBNP.

### BACKGROUND

Congestive heart failure (CHF) (or commonly “heart failure”) is a chronic and widespread condition in which a heart cannot pump well enough to meet the body's needs. There are two types of CHF: heart failure with reduced ejection fraction left ventricular function (HF-rEF)/Diastolic HF; and heart failure with preserved ejection fraction (HF-pEF)/Systolic HF.

Known biomarkers of CHF are brain (or B-type) natriuretic peptide (BNP) and associated N-terminal brain natriuretic peptide (NT-proBNP). These peptides are the product of the cleavage of the prohormone proBNP, which is produced by the heart. BNP and NT-proBNP are used as the primary indicators of CHF (American Heart Association Guidelines). Most assays look at the concentration of BNP and/or NT-proBNP in the blood. A review article by Semenov and Feygina (Adv Clin Chem. 2018; 85: 1-30) provides an overview of available assays. Of note, the assays mentioned in the review either cannot discriminate between pro-BNP and BNP or NT-BNP or are used in detecting only one of the three natriuretic peptides.

Sole reliance on BNP and/or NT-proBNP to diagnose or monitor CHF can lead to inaccuracies. Existing immunoassays lack adequate precision and discriminative accuracy of specific biomarker differentiation, among BNP vs proBNP vs NT-proBNP because they usually target epitopes that are common among one or more of the three natriuretic peptides, leading to assay pollution. Moreover, the half-life of each of these peptides is different. Specifically, the half-life of NT-proBNP is approximately 6 times that of BNP. This can lead to difficulty in determining the effectiveness of therapy administered to a patient suffering from heart disease and particularly heart failure, e.g., in the context of angiotensin receptor-neprilysin inhibitor (ARNI) therapy which specifically targets and modulates BNP levels and not NT-proBNP.

Dries and colleagues (Circ Heart Fail. 2010; 3(2): 220-227) suggested that simultaneous assessment of unprocessed proBNP in addition to processed BNP could improve the identification of high-risk ambulatory patients with heart failure. They did not assay NT-proBNP. Moreover, they employed an assay for measuring BNP that demonstrates some cross-reactivity with proBNP.

Monitoring CHF can be difficult, particularly in a non-clinical environment. In clinical environments, a BNP/NT-proBNP measurement is performed using a venous puncture-based laboratory test, which takes several hours for results to be determined. In a point-of-care setting, venous or capillary blood samples are taken, thus still requiring clinical care for execution.

Accordingly, there is a lack of a continuous and reliable measurement of natriuretic peptides for the purpose of heart failure diagnosis and management, and particularly one that can be used easily in a point-of-care setting.

### SUMMARY OF THE DISCLOSURE

The present disclosure provides devices for determining the presence of BNP, proBNP and NT-proBNP, allowing a more accurate diagnosis and monitoring of CHF. The devices comprise multiple capture regions configured to selectively capture BNP, proBNP and NT-proBNP. The capture regions are arranged in such a manner that assay pollution by proBNP is minimized. For example, by capturing proBNP first and removing it from the sample, assay pollution during the subsequent detection of BNP and NT-proBNP may be avoided. Alternatively, or additionally, the devices may employ distinct epitopes for labelling and capturing proBNP so that it can be distinguished from NT-proBNP and/or BNP, thereby improving the accuracy of detection.

In certain embodiments, a device for determining the presence of BNP, proBNP and NT-proBNP in a sample is provided, the device comprising:
(i) a first capture region comprising a first capture species, wherein the first capture species binds to an epitope that is located within a region defined by amino acids 72-105 of SEQ ID NO: 2, wherein the epitope either comprises amino acids 75-78 of SEQ ID NO: 2, or comprises amino acids located on either side of a cleavage site between amino acid 76 and amino acid 77 of SEQ ID NO: 2, wherein said first capture species specifically binds to proBNP, but does not bind NT-proBNP or BNP;
(ii) a second capture region comprising a second capture species that binds to an epitope comprising amino acids within 87-97 of SEQ ID NO: 2; and
(iii) a third capture region comprising a third capture species that binds to an epitope within a region comprising amino acids 10-76 of SEQ ID NO: 2.

In certain embodiments, a device for determining the presence of BNP, proBNP and NT-proBNP in a sample is provided, the device comprising:
(i) a first capture region comprising a first capture species, wherein the first capture species binds to an epitope comprising amino acids 72-85 of SEQ ID NO: 2, wherein said first capture species specifically binds to proBNP, but does not bind NT-proBNP or BNP;
(ii) a second capture region comprising a second capture species that binds to an epitope comprising amino acids within 87-97 of SEQ ID NO: 2; and
(iii) a third capture region comprising a third capture species that binds to an epitope within a region comprising amino acids 10-76 of SEQ ID NO: 2.

In certain embodiments, a device for determining the presence of BNP, proBNP and NT-proBNP in a sample is provided, the device comprising:
(i) a first capture region comprising a first capture species binds to a conformational epitope comprising amino acids within (a) a first region defined by amino acids 72-85 of SEQ ID NO: 2 and (b) a second region defined by amino acids 98-105 of SEQ ID NO: 2; wherein said first capture species specifically binds to proBNP, but does not bind NT-proBNP or BNP;
(ii) a second capture region comprising a second species that binds to an epitope comprising amino acids within 87-97 of SEQ ID NO: 2; and
(iii) a third capture region comprising a third capture species that binds to an epitope within a region comprising amino acids 10-76 of SEQ ID NO: 2.

In certain embodiments, a device for determining the presence of BNP, proBNP and NT-proBNP in a sample is provided, the device comprising:
(i) a reservoir region comprising a first detection species, wherein the first detection species comprises a first label detectable to produce a first signal and binds to either (a) a first epitope within a region comprising amino acids 10-76 of SEQ ID NO: 2 or (b) a second epitope within a region comprising amino acids 77-108 of SEQ ID NO: 2;
(ii) a first capture region comprising a first capture species that binds to the other of (a) a first epitope within a region comprising amino acids 10-76 of SEQ ID NO: 2 or (b) a second epitope within a region comprising amino acids 77-108 of SEQ ID NO: 2, such that one of the first capture species and the first detection species binds to the first epitope and the other of the first capture species and the first detection species binds to the second epitope;
(iii) a second capture region comprising a second capture species, wherein the second capture species binds to a third epitope located within the same region as the epitope bound by the first detection species; and
(iv) optionally a third capture region comprising a third capture species, wherein the third capture species binds to a fourth epitope located within the same region as the epitope bound by the first capture species,
wherein the device is configured such that binding of BNP and proBNP in the first capture region produces a second signal, which second signal is distinguishable from the first signal.

### BRIEF DESCRIPTION OF THE DRAWINGS

The present disclosure will now be described in more detail with reference to the accompanying drawings, which are not intended to be limiting:

FIG. 1 provides a schematic plan view of a lateral flow assay according to an embodiment;

FIG. 2 provides a schematic plan view of another lateral flow assay according to an embodiment;

FIG. 3 provides a schematic plan view of an electrode structure according to an embodiment;

FIG. 4 provides a schematic plan view of another device according to an embodiment;

FIG. 5 provides a schematic plan view of another device according to an embodiment; and

FIG. 6 provides a schematic plan view of another device according to an embodiment.

### DETAILED DESCRIPTION

### General Issues

Congestive heart failure (CHF) (or commonly “heart failure”) is a chronic and widespread condition in which a patient's heart cannot pump well enough to meet the body's needs. There are two types of CHF: heart failure with reduced left ventricular function (HF-rEF)/Diastolic HF; and heart failure with preserved left ventricular function (HF-pEF)/Systolic HF.

Known biomarkers of CHF are brain (or B-type) natriuretic peptide (BNP) and associated N-terminal brain natriuretic peptide (NT-proBNP). These natriuretic peptides are the product of the cleavage of the prohormone proBNP, which is produced by the heart. BNP and NT-proBNP are used as the primary indicators of CHF (American Heart Association Guidelines). Most assays will look at the concentration of BNP and/or NT-proBNP in the blood. However, reliance on these two biomarkers (BNP and NT-proBNP) can lead to inaccuracies due to the presence of proBNP. This can lead to difficulty in determining the effectiveness of therapy administered, for e.g., angiotensin receptor-neprilysin inhibitor (“ARNI”) which specifically targets and modulates BNP levels.

Moreover, monitoring CHF can be difficult, particularly in a non-clinical environment. There is a lack of a continuous and reliable measurement of natriuretic peptides for the purpose of heart failure diagnosis and management.

BNP is secreted by the heart through the coronary sinus, predominantly from the cardiac ventricles. The pre-pro peptide precursor of BNP (pre-proBNP) is 134 amino acids in length:

```
(SEQ ID NO: 1)
                10 20 30 40
MDPQTAPSRA LLLLLFLHLA FLGGRSHPLG SPGSASDLET
                50 60 70 80
SGLQEQRNHL QGKLSELQVE QTSLEPLQES PRPTGVWKSR
                90 100 110 120
EVATEGIRGH RKMVLYTLRA PRSPKMVQGS GCFGRKMDRI
              130
SSSSGLGCKV LRRH
```

It comprises a short signal peptide (underlined above), which is enzymatically cleaved off to release the human 108 amino acid long pro-peptide of BNP (proBNP):

```
(SEQ ID NO: 2)
                10 20 30 40 50
HPLGSPGSAS DLETSGLQEQ RNHLQGKLSE LQVEQTSLEP LQESPRPTGV
                60 70 80 90 100
WKSREVATEG IRGHRKMVLY TLRAPRSPKM VQGSGCFGRK MDRISSSSGL
                                                                        ↑
GCKVLRRH
```

The enzyme corin cleaves proBNP into a 76 amino acid long N-terminal pro-peptide of BNP (NT-proBNP; SEQ ID NO:3) and the 32 amino acid long peptide hormone BNP (SEQ ID NO:4):

```
(SEQ ID NO: 3)
                10 20 30 40
HPLGSPGSAS DLETSGLQEQ RNHLQGKLSE LQVEQTSLEP
                50 60 70
LQESPRPTGV WKSREVATEG IRGHRKMVLY TIRAPR
(SEQ ID NO: 4)
SPKMVQGSGC FGRKMDRISS SSGLGCKVLR RH
```

The cleavage site is indicated by an arrow in the amino acid sequence of SEQ ID NO:2. BNP has a very short half-life of only about 20 minutes. It is degraded by neprilysin, a membrane metallo-endopeptidase.

proBNP and BNP contain a loop structure which is formed by a stretch of amino acids (underlined in the proBNP and BNP sequences above) through a disulfide bond between two cysteine residues (shown in bold in the proBNP and BNP sequences above).

Conventional diagnostic assays commonly use antibodies to detect BNP, and may also detect NT-proBNP. Such assays typically fail to account for the precursor peptide proBNP. As BNP is a cleavage product of proBNP, the presence of free unbound proBNP may result in assay pollution during the detection of BNP. In order to improve assay accuracy, the present disclosure provides, among other things, devices employing capture species (e.g., antibodies). The devices and assays provided herein can differentiate between proBNP, BNP and NT-proBNP.

In particular, the present disclosure provides a device for determining the presence of BNP, proBNP and NT-proBNP in a sample, wherein the device comprises:
(i) a first capture region comprising a first capture species, wherein the first capture species binds to an epitope that is located within a region defined by amino acids 72-105 of SEQ ID NO: 2, wherein the epitope either comprises amino acids 75-78 of SEQ ID NO: 2, or comprises amino acids located on either side of a cleavage site between amino acid 76 and amino acid 77 of SEQ ID NO: 2, wherein said first capture species specifically binds to proBNP, but does not bind NT-proBNP or BNP;
(ii) a second capture region comprising a second capture species that binds to an epitope comprising amino acids within 87-97 of SEQ ID NO: 2; and
(iii) a third capture region comprising a third capture species that binds to an epitope within a region comprising amino acids 10-76 of SEQ ID NO: 2.

The present disclosure also provides a device for determining the presence of BNP, proBNP and NT-proBNP in a sample, wherein the device comprises:
(i) a first capture region comprising a first capture species, wherein the first capture species binds to an epitope comprising amino acids 72-85 of SEQ ID NO: 2, wherein said first capture species specifically binds to proBNP, but does not bind NT-proBNP or BNP;
(ii) a second capture region comprising a second capture species that binds to an epitope comprising amino acids within 87-97 of SEQ ID NO: 2; and
(iii) a third capture region comprising a third capture species that binds to an epitope within a region comprising amino acids 10-76 of SEQ ID NO: 2.

The present disclosure further provides a device for determining the presence of BNP, proBNP and NT-proBNP in a sample is provided, wherein the device comprises:
(i) a first capture region comprising a first capture species binds to a conformational epitope comprising amino acids within (a) a first region defined by amino acids 72-85 of SEQ ID NO: 2 and (b) a second region defined by amino acids 98-105 of SEQ ID NO: 2; wherein said first capture species specifically binds to proBNP, but does not bind NT-proBNP or BNP;
(ii) a second capture region comprising a second species that binds to an epitope comprising amino acids within 87-97 of SEQ ID NO: 2; and
(iii) a third capture region comprising a third capture species that binds to an epitope within a region comprising amino acids 10-76 of SEQ ID NO: 2.

The present disclosure also provides a device for determining the presence of BNP, proBNP and NT-proBNP in a sample is provided, wherein the device comprises:
(i) a reservoir region comprising a first detection species, wherein the first detection species comprises a first label detectable to produce a first signal and binds to either (a) a first epitope within a region comprising amino acids 10-76 of SEQ ID NO: 2 or (b) a second epitope within a region comprising amino acids 77-108 of SEQ ID NO: 2;
(ii) a first capture region comprising a first capture species that binds to the other of (a) a first epitope within a region comprising amino acids 10-76 of SEQ ID NO: 2 or (b) a second epitope within a region comprising amino acids 77-108 of SEQ ID NO: 2, such that one of the first capture species and the first detection species binds to the first epitope and the other of the first capture species and the first detection species binds to the second epitope;
(iii) a second capture region comprising a second capture species, wherein the second capture species binds to a third epitope located within the same region as the epitope bound by the first detection species; and
(iv) optionally a third capture region comprising a third capture species, wherein the third capture species binds to a fourth epitope located within the same region as the epitope bound by the first capture species,
   wherein the device is configured such that binding of BNP and proBNP in the first capture region produces a second signal, which second signal is distinguishable from the first signal.

Suitable capture and detection species (e.g., antibodies) that can be employed in a device in accordance with the present disclosure are readily available. The following sections describe exemplary antibodies that may be used in the capture and/or detection of BNP, proBNP and NT-proBNP.

proBNP Capture and Detection

A key feature of the present disclosure is to capture proBNP in an initial step of the detection assay (e.g., using a device in accordance with the present disclosure) to avoid downstream pollution during the detection of BNP or NT-proBNP. Nuanced differentiation of the three peptides is crucial for monitoring heart failure (specifically HF-rEF but also to some extent HF-pEF) patients.

proBNP-Specific Capture Species A capture species used for the capture of proBNP in accordance with the present disclosure specifically binds to proBNP, but does not bind NT-proBNP or BNP. Typically, this means that the capture species has an at least 100-fold higher binding affinity for proBNP than either NT-proBNP or BNP. More typically, the capture species has an at least 1000-fold higher binding affinity for proBNP than either NT-proBNP or BNP.

In some embodiments, a capture species used for the capture of proBNP binds to an epitope that is located within a region defined by amino acids 72-105 of SEQ ID NO: 2 and spans the site at which proBNP is cleaved into NT-proBNP and BNP, i.e., the epitope either comprises amino acids surrounding the cleavage site, or comprises amino acids on either side of the cleavage site, which is located between amino acid 76 and amino acid 77 of SEQ ID NO: 2 (but does not necessarily include amino acids that are located proximal to the cleavage site including amino acids 76 and 77).

For example, in one embodiment, a capture species (e.g., an antibody) used for the capture of proBNP binds to an epitope comprising amino acids 75-79 of SEQ ID NO: 2, which includes the site at which proBNP is cleaved into NT-proBNP and BNP. In some embodiments, a capture species used for the capture of proBNP binds to an epitope comprising amino acids 72-85 of SEQ ID NO: 2. This epitope also comprises the site at which proBNP is cleaved into NT-proBNP and BNP.

In another embodiment, the capture species (e.g., an antibody) binds two or more amino acids that are located on either side of the site at which proBNP is cleaved into NT-proBNP and BNP within a region defined by amino acids 72-105 of SEQ ID NO: 2. Such an epitope may not include amino acids that are located proximal to the cleavage site (including amino acids 76 and 77), e.g., it may be a conformational epitope. For example, in some embodiments, a capture species used for the capture of proBNP binds to an epitope comprising amino acids within (i) a first region defined by amino acids 72-76 of SEQ ID NO: 2 and (ii) a second region defined by amino acids 77-85 of SEQ ID NO: 2. In other embodiments, a capture species used for the capture of proBNP binds to an epitope comprising amino acids within (i) a first region defined by amino acids 72-76 of SEQ ID NO: 2 and (ii) a second region defined by amino acids 86-101 of SEQ ID NO: 2. In further embodiments, a capture species used for the capture of proBNP binds to a conformational epitope comprising amino acids within (i) a first region defined by amino acids 72-85 of SEQ ID NO: 2 and (ii) a second region defined by amino acids 98-105 of SEQ ID NO: 2. Binding to an epitope that includes the site at which proBNP is cleaved into NT-proBNP and BNP aids specificity for proBNP. Moreover, the N and C termini of BNP are prone to detection but remain intact in proBNP. Accordingly, a capture species that also binds to amino acids 98-105 of proBNP (SEQ ID NO: 2), which may be absent in BNP, may further increase specificity.

A suitable antibody that binds to an epitope that is located within a region defined by amino acids 72-105 of SEQ ID NO: 2 and spans the site at which proBNP is cleaved into NT-proBNP and BNP is the “Hinge76” antibody disclosed in Giuliani et al. (Clin Chem. 2006; 52(6): 1054-61). This monoclonal antibody specifically binds to proBNP, but does not bind NT-proBNP or BNP. It binds a conformational epitope that comprises amino acids within (i) a first region defined by amino acids 72-76 of SEQ ID NO: 2 and (ii) a second region defined by amino acids 77-85 of SEQ ID NO: 2. The Giuliani paper also describes a method of obtaining further antibodies that meet the requirements of the claimed present disclosure.

proBNP Detection

In some embodiments, a detection species for the detection of proBNP binds to the same epitope bound by the detection species used for the detection NT-proBNP and/or BNP. In some embodiments, the same detection species (e.g., the same antibody) is used for the detection of proBNP and NT-proBNP. In other embodiments, the same detection species (e.g., the same antibody) is used for the detection of proBNP and BNP.

In some embodiments, a detection species used for the detection of proBNP binds to an epitope that is distinct from the epitopes bound by the detection species used for the detection NT-proBNP and BNP.

Employing Distinct Epitopes for Labelling and Capturing proBNP

Alternatively, assay accuracy can be improved by the manner employed for detecting proBNP relative to NT-proBNP and BNP. For example, a capture species may be used that binds to an epitope present in both proBNP and BNP. For instance, the capture species used for the capture of proBNP may bind to an epitope within a region comprising amino acids 77-108 of SEQ ID NO: 2. In some embodiments, the capture species binds to an epitope within a region comprising amino acids 77-108 of SEQ ID NO: 2. In some embodiments, the capture species binds to an epitope comprising amino acids 102-108 of SEQ ID NO: 2. In order to specifically detect proBNP, a detection species is used that binds to an epitope within a region comprising amino acids 10-76 of SEQ ID NO: 2. In some embodiments, the detection species binds to an epitope comprising amino acids 13-20 of SEQ ID NO: 2. As this region is absent in BNP, only proBNP is detected. Moreover, the same detection species can be used to detect both proBNP and NT-proBNP. However, in the case of NT-proBNP, the capture species for capturing the proBNP will not capture NT-proBNP and hence these can be distinguished.

The reverse configuration is also possible, whereby a capture species may be used that binds to an epitope present in both proBNP and NT-proBNP. For instance, the capture species used for the capture of proBNP may bind to an epitope within a region comprising amino acids 10-76 of SEQ ID NO: 2. In order to specifically detect proBNP, a detection species is used that binds to an epitope within a region comprising amino acids 77-108 SEQ ID NO: 2. As this region is absent in NT-proBNP, only proBNP is detected.

Suitable capture and detection species (e.g., antibodies) to increase assay accuracy in the manner described in the preceding paragraphs are readily available. For example, Seferian et al. (Clin Chem. 2007 May; 53(5):866-73; which is incorporated herewith by reference) provide antibodies that bind to epitopes comprising amino acids amino acids 13-20 and 102-108 of SEQ ID NO: 2, referred to therein as monoclonal antibodies 16F3 and 50E1, respectively. The Seferian paper also describes methods of making such antibodies. Antibodies 16F3 and 50E1 can be purchased from HyTest, Turku, Finland.

### BNP Capture and Detection

The 32 amino acid long BNP may be subject to both C- and N-terminal degradation. Accordingly, in typical embodiments, capture species used for the detection of BNP in a sample target the stable loop region of BNP (also referred to as “the ringed structure”). The stable loop region comprises amino acids 11-25 of BNP (SEQ ID NO: 4), which correspond to amino acids 87-101 of SEQ ID NO: 2. In some embodiments, a capture species (e.g., an antibody) binds to an epitope with the loop region. In some embodiments, the capture species binds to an epitope within a region comprising amino acids 11-21 of BNP (SEQ ID NO: 4), which correspond to amino acids 87-97 of SEQ ID NO:2.

The stable loop region is also present in proBNP. In a typical embodiment of the present disclosure, a sample is first exposed to a capture species (e.g., an antibody) used for the capture of proBNP, thereby removing it from the sample. Accordingly, it is not necessary that a capture species (e.g., an antibody) used for the capture of BNP is capable of discriminating between proBNP and BNP.

In some embodiments, a capture species used in the detection of BNP binds to an epitope comprising amino acids 11-17 of BNP (SEQ ID NO: 4), which correspond to amino acids 87-93 of SEQ ID NO: 2. In a specific embodiment, the capture species binds to an epitope consisting of amino acids 11-17 of BNP. In some embodiments, the capture species is an antibody, e.g., a monoclonal antibody (mAb). In particular embodiments, the capture antibody is mAb 24C5.

In some embodiments, a detection antibody used for the detection of BNP binds to a complex formed by the capture antibody and BNP. For example, the complex formed by capture antibody mAb 24C5 and BNP is bound by Ab-BNP2. Accordingly, in some embodiments, the detection antibody used for the detection of BNP is Ab-BNP2.

In some embodiments, a capture species used in the detection of BNP binds to an epitope comprising amino acids 11-22 of BNP (SEQ ID NO: 4), which correspond to amino acids 87-98 of SEQ ID NO: 2. In a specific embodiment, the capture species binds to an epitope consisting of amino acids 11-22 of BNP. In some embodiments, the capture species is an antibody, e.g., a monoclonal antibody (mAb). In particular embodiments, the capture antibody is mAb 26E2.

In other embodiments, a capture species used in the detection of BNP binds to an epitope comprising amino acids 14-21 of BNP (SEQ ID NO: 4), which correspond to amino acids 90-97 of SEQ ID NO: 2. In a specific embodiment, the capture species binds to an epitope consisting of amino acids 14-21 of BNP. In some embodiments, the capture species is an antibody, e.g., a monoclonal antibody (mAb).

In some embodiments, a detection species used in the detection of BNP bind to an epitope comprising amino acids 26-32 of BNP (SEQ ID NO: 4), which correspond to amino acids 102-108 of SEQ ID NO: 2. In some embodiments, the detection species is an antibody, e.g., a monoclonal antibody (mAb).

In further embodiments, a capture species used in the detection of BNP binds to an epitope comprising amino acids 27-32 of BNP (SEQ ID NO: 4), which correspond to amino acids 103-108 of SEQ ID NO: 2. In other embodiments, a detection species used in the detection of BNP binds to an epitope comprising amino acids 14-21 of BNP (SEQ ID NO: 4), which correspond to amino acids 90-97 of SEQ ID NO: 2. In some embodiments, the capture and detection species are antibodies, e.g., monoclonal antibodies.

In further embodiments, a capture species used in the detection of BNP binds to an epitope comprising amino acids 5-15 of BNP (SEQ ID NO: 4), which correspond to amino acids 81-91 of SEQ ID NO: 2. In other embodiments, a detection species used in the detection of BNP binds to an epitope comprising amino acids 26-32 of BNP (SEQ ID NO: 4), which correspond to amino acids 102-108 of SEQ ID NO: 2. In some embodiments, a capture antibody is anti-BNP (106.3), and a detection antibody is anti-BNP (BC203) for use according to the present disclosure. Abbott's Architect and Alinity i BNP assays employ 106.3 and BC203 to measure BNP.

Several commercially available BNP immunoassays use antibodies that are specific for epitopes in the stable loop region (e.g., AxSYM, Centaur, Dimension, Access and Triage). Such antibodies are readily available. Monoclonal antibodies 24C5, 26E2 and Ab-BNP2 can be purchased from HyTest, Turku, Finland.

### NT-proBNP Capture and Detection

proBNP and NT-proBNP can be glycosylated, and it is typically these glycosylated forms that are present in samples obtained from a patient. There are seven glycosylation sites located in a 36-amino acid region within the N-terminal portion of proBNP and its cleavage product NT-proBNP (amino acids 36-71 of SEQ ID NO: 2 and SEQ ID NO:3).

In some embodiments, a non-terminal and non-glycosylated epitope on NT-proBNP is used to capture NT-proBNP. Suitable capture species that bind such epitopes on NT-proBNP are readily available for use in the present disclosure. In some embodiments, the capture species is an antibody, e.g., a monoclonal antibody (mAb).

For example, in some embodiments, the capture species (e.g., an antibody) used for the capture of NT-proBNP binds to an epitope within a region comprising amino acids 5-12 of NT-proBNP, which correspond to amino acids 5-12 of SEQ ID NO: 2. An exemplary antibody that binds an epitope within this region is the monoclonal antibody 29D12. In some embodiments, the capture species (e.g., an antibody) used for the detection of NT-proBNP may bind to an epitope within a region comprising amino acids 13-20 of NT-proBNP, which correspond to amino acids 13-20 of SEQ ID NO: 2. An exemplary antibody that binds an epitope within this region is the monoclonal antibody 13G12. In some embodiments, the capture species (e.g., an antibody) used for the detection of NT-proBNP binds to an epitope within a region comprising amino acids 27-31 of NT-proBNP, which correspond to amino acids 27-31 of SEQ ID NO: 2. In some embodiments, the capture species (e.g., an antibody) binds to an epitope consisting of amino acids 27-31 of NT-proBNP. For example, the capture species may be an antibody (e.g., a monoclonal antibody) that binds to an epitope comprising amino acids 25-32 of SEQ ID NO: 2. An exemplary antibody that binds an epitope within this region is the monoclonal antibody mAb NT34.

In other embodiments, the capture species used for the capture of NT-proBNP may bind to an epitope within a region containing one or more glycosylation sites. In some embodiments, the capture species (e.g., an antibody) used for the capture of NT-proBNP binds to an epitope within a region comprising amino acids 63-71 of SEQ ID NO: 2. An exemplary antibody that binds this region is the monoclonal antibody 15C4.

Typically, the capture species used for capturing NT-proBNP and the detection species used for detecting NT-proBNP bind to distinct epitopes on NT-proBNP. In some embodiments, the detection species (e.g., an antibody) binds a non-terminal and non-glycosylated epitope on NT-proBNP. In other embodiments, the detection species (e.g., an antibody) binds an epitope within a region containing one or more glycosylation sites.

In some embodiments, the detection species used for the detection of NT-proBNP binds to an epitope comprising amino acids 42-46 of SEQ ID NO: 2, which correspond to amino acids 42-46 of SEQ ID NO: 2. In a specific embodiment, the detection species binds to an epitope consisting of amino acids 42-46 of NT-proBNP.

For example, the capture species used for the capture of NT-proBNP may bind to an epitope within a region comprising amino acids 27-31 of SEQ ID NO: 2, and the detection species used for the detection of NT-proBNP may bind to an epitope within a region comprising amino acids 42-46 of SEQ ID NO: 2. In some embodiments, the capture species used for the capture of NT-proBNP binds to an epitope within a region comprising amino acids 27-31 of SEQ ID NO: 2, and the detection species used for the detection of NT-proBNP binds to an epitope within a region comprising amino acids 63-71 of SEQ ID NO: 2.

Commercially available antibodies that target NT-proBNP can be used as capture and detection species in practicing the present disclosure. In some embodiments, the capture antibody binds to an epitope within a region comprising amino acids 63-71 of SEQ ID NO: 2 (e.g., mAb 15C4), and the detection antibody binds to an epitope within a region comprising amino acids 13-20 of SEQ ID NO: 2 (e.g., mAb 13G12). In some embodiments, the capture antibody binds to an epitope within a region comprising amino acids 63-71 of SEQ ID NO: 2 (e.g., mAb 15C4), the detection antibody binds to an epitope within a region comprising amino acids 25-32 of SEQ ID NO: 2 (e.g., mAb NT34). In other embodiments, the capture antibody binds to an epitope within a region comprising amino acids 5-12 of SEQ ID NO: 2 (e.g., mAb 29D12), and the detection antibody binds to an epitope within a region comprising amino acids 25-32 of SEQ ID NO: 2 (e.g., mAb NT34).

In some embodiments, the capture antibody binds to an epitope within a region comprising amino acids 27-31 of SEQ ID NO: 2 (e.g., a murine monoclonal antibody), and the detection antibody binds to an epitope within a region comprising amino acids 42-46 of SEQ ID NO: 2 (e.g., a sheep monoclonal antibody specific to a partially glycosylated region of NT-proBNP). A commercial NT-proBNP assay employing such capture and detection antibodies is manufactured by Roche.

### Use of the Assay of the Present Disclosure

The assays, methods and devices of the present disclosure are used for the detection of proBNP, BNP and NT-proBNP in a sample. A typical sample obtained from a patient comprises blood, plasma, serum or saliva.

Most assays look at the concentration of BNP and NT-proBNP in a patient sample as primary indications of congestive heart failure (CHF). However, reliance on these two biomarkers (BNP and NT-proBNP) can lead to inaccuracies.

The nuanced differentiation of the three peptides proBNP, BNP and NT-proBNP is crucial for monitoring heart function (specifically in patients suffering from HF-rEF but also to some extent in patients suffering HF-pEF). The assays according to the present disclosure provide selectivity which can distinguish these three analytes.

The diagnostic assay of the present disclosure may be particularly useful in monitoring heart disease in patients who are on Angiotensin Receptor and Neprilysin Inhibitor (ARNI) therapy. ARNI results in neprilysin inhibition and therefore may lead to a drug-induced raise in BNP levels, whereas NT-proBNP levels remain unaffected. In particular, the proposed assays and methods make a high probability, high accuracy differentiation between BNP and proBNP levels.

Additionally, monitoring CHF can be difficult, particularly in a non-clinical environment. In current clinical practice, BNP/NT-proBNP measurement is performed either using a venous puncture-based laboratory test (takes several hours to get results) or in a point-of-care setting using venous or capillary blood samples, nevertheless needing a clinical care location for execution. Traditional laboratory tests are time consuming and require phlebotomy or venous puncture for blood draw and are often too slow for treating acute CHF. The use of the devices disclosed herein, such as a flow device (e.g., a lateral flow device) and/or an electrochemical sensor provides a straightforward assay for measuring these levels.

### Detection Systems

Detection of the analytes (i.e., proBNP, BNP and NT-proBNP) when bound to the capture analytes may be carried out in any manner known in the art. In some embodiments, the detection may comprise the use of tags or labels, for example attached to detection species (e.g., antibodies) configured to bind to epitopes of the BNP, proBNP and NT-proBNP fragments. These may bind to epitopes separate to those of the capture species so as to not interfere with capture. In other embodiments, label-free detection methods may be used (such as electrochemical detection). Detection methods, whether label-dependent or label-free, can include optical, surface plasma resonance, electrical, electrochemical, magnetic, and chemical image processing methods.

Accordingly, in some embodiments, the present disclosure provides a device comprising a sample-receiving region; and a reservoir region comprising a first detection species, a second detection species and a third detection species, wherein the first and third detection species each bind to a distinct epitope on proBNP as defined by SEQ ID NO:2, wherein the distinct epitopes bound by the first and third detection species are different from each of the epitopes bound by the first, second and third capture species. These embodiments may be in the form of a lateral flow test. In such embodiments, these components may be provided on a substrate, such as a nitrocellulose membrane. These provide rapid and relatively cheap methods of determining the presence and quantity of the analytes in the sample.

### Lateral Flow Assay

A typical lateral flow assay assembly comprises a sample receiving region and three capture regions arranged sequentially—a first capture region comprising a first capture species, a second capture region comprising a second capture species and a third capture region comprising a third capture species. The capture regions are arranged in a manner that when a sample is added to the assembly at the sample receiving region, the direction of flow of the sample through the assembly is such that the first capture region captures full-length proBNP, the second capture region captures NT-proBNP, and the third capture region captures BNP.

Specifically, the first capture species specifically binds proBNP, but does not bind BNP or NT-proBNP. The second capture species binds to NT-proBNP, but not BNP. Accordingly, BNP passes through the first and second capture regions unimpededly and is captured by the third capture species in the third capture region. Without wishing to be bound by any particular theory, an advantage of arranging the three capture regions in this manner on the lateral flow assay assembly is that proBNP, NT-proBNP and BNP may reach their respective capture regions at the same time.

A suitable first capture species (e.g., an antibody) may bind to an epitope comprising amino acids 72-85 of SEQ ID NO: 2 (e.g., including the site at which proBNP is cleaved into NT-proBNP and BNP). Alternatively, a suitable first capture species binds to a conformational epitope comprising amino acids within (i) a first region defined by amino acids 72-85 of SEQ ID NO: 2 and (ii) a second region defined by amino acids 98-105 of SEQ ID NO: 2. A suitable second capture species binds to an epitope comprising amino acids 27-31 of SEQ ID NO: 2. A suitable third capture species binds to an epitope comprising amino acids amino acids 87-93 of SEQ ID NO: 2.

In an alternative embodiment, the second and third capture regions may be reversed, such that capture regions are arranged in a manner that when a sample is added to the assembly at the sample receiving region, the direction of flow of the sample through the assembly is such that the first capture region captures full-length proBNP, the next capture region (designated the third capture region) captures BNP, and the final capture region, sequentially, (designated the second capture region) captures NT-proBNP.

### Electrochemical Detection

In certain embodiments, the device uses electrochemical detection to detect the presence of BNP, proBNP or NT-proBNP. Electrochemical sensors use electrodes (test or working electrodes) with a capture species bound to the surface of the working or test electrodes. In certain embodiments, the first capture region comprises a first electrode set comprising at least one electrode, wherein the first capture species is bound to a surface of the electrode of the first electrode set; the second capture region comprises a second electrode set comprising at least one electrode, wherein the second capture species is bound to a surface of the electrode of the second electrode set; and the third capture region comprises a third electrode set comprising at least one electrode, wherein the third capture species is bound to a surface of the electrode of the third electrode set.

In some embodiments, the species selectively interacting with, for example binding, the target epitopes(s) causes a change in the signal produced by the respective electrode. For example, there may be a change in the potential of the electrode.

In some embodiments, the species are functionalized with an electro-active moiety, for example a redox-active moiety. Any suitable electro-active moiety may be used for this purpose, such as methylene blue. Upon selectively interacting with, for example binding, the target epitopes(s) causes a change in the proximity of the electro-active moiety with respect to the surface of the respective electrode, leading to a change in signal. In some embodiments, an external field can be applied (such as an electrical or magnetic field) to manipulate the bound analytes so as to modify the relative position of tags or labels relative to the electrode surface. The response can provide an indication of the size of the bound entity, providing a further confirmation of the bound species. The molecular weight of each of proBNP>NT-proBNP>BNP when bound to the respective species creates specific signatures.

Binding or adhering the electrode surfaces with the respective capture species (i.e., functionalizing the surface) can be achieved in any suitable manner, such as by covalently or non-covalently immobilizing the capture species to the surface. For example, these can be immobilized, for example grafted, onto the surface of a noble metal, for example gold or platinum, electrode.

Where the device is an electrochemical sensor with functionalized electrodes, the functionalization of each of the electrodes to define the first, second and third capture regions with the capture species in this ratio may be achieved by varying the density of the coverage of the capture species and/or the area of the electrodes covered by the capture species. For example, in some embodiments, there may be a uniform coverage of species on each electrode set but the area may be adjusted to provide the different ratios. Uniform coverage may permit the same protocol for functionalizing the sets of electrode(s), for example using the same reagent drop size, reagents, washes, etc. Alternatively or additionally, the density of the capture species on the electrode surfaces may be varied to provide the different ratios. Varying the density of the capture species may be implemented in any suitable manner, such as by varying the concentration of the capture species solution used for functionalizing the surfaces. A more concentrated solution may provide a higher density, for example packing density, of the capture species on the surface of the respective electrode.

Accordingly, in some embodiments, the binding of BNP, proBNP and NT-proBNP is directly guided by the relative number of capture species available for each peptide sequence when binding is diffusion-based binding. The highest chance to bind is given to the most highly available peptide sequence. Moreover, the geographical separation of the capture species ensures that corresponding amplifiers record the appropriate signals for BNP, proBNP or NT-proBNP, respectively.

Without wishing to be bound by any particular theory, the currents detected by the electrodes may exhibit minute time-constant differences in response to binding of a larger peptide such as proBNP as opposed to shorter peptides such as NT-proBNP and BNP. Accordingly, a flow-based system may not be required because the shorter peptides travel faster and bind appropriate species before the larger proBNP peptide reaches that capture region, in particular when the capture regions are sized to the expected ratios of each of BNP, proBNP or NT-proBNP (see below).

The devices in the embodiments employing electrochemical sensing principles may comprise an electrochemical cell comprising the electrode sets defined herein and further a counter electrode. The devices may thus be configured for determining a change in current associated with the selective interaction of the capture species with their respective epitopes. The counter electrode may act as a cathode or anode to the working electrodes, for example the electrode sets. In some embodiments, the devices may further comprise a reference electrode. The reference electrode may constitute a site of a known chemical reaction having a known redox potential. Any suitable reference electrode may be employed, such as a saturated calomel, silver/silver chloride, or a copper/copper sulfate reference electrode.

### Ratios of Capture Species

In some embodiments, the first, second and third capture species in the first, second and third capture regions are present at a ratio of about 1.x:1:6, where x=0 to 9, for example 1.5:1:6. This corresponds to an expected ratio of each of proBNP, BNP and NT-proBNP in the sample and thus provides an appropriate array of capture regions. This may be provided in conjunction with any of the devices and detection methods set out above.

### Capture Species and Detection Species

The devices, systems and methods disclosed herein utilize analyte binding technology. A capture species with specificity for the particular analyte, in this context an epitope, can be used to bind the analyte (i.e., BNP, proBNP or NT-proBNP).

Any suitable capture species can be selected for this purpose.

In some embodiments, the capture species may comprise an antibody which is adapted to bind to the recited epitopes, or a part thereof (i.e., amino acids within the recited epitopes). Thus, the first, second and third capture species may each be first, second and third capture antibodies.

More generally, the capture species may, in some embodiments, comprise at least one selected from a protein, a peptide, a carbohydrate, and a nucleic acid.

In an embodiment, the capture species comprises an aptamer. An aptamer may be defined as an oligonucleotide configured to bind the epitope of interest.

In some non-limiting examples, the aptamer is functionalized with an electro-active moiety, for example a redox-active moiety, and is configured such that a conformational change of the aptamer upon selectively interacting with, for example binding, the analyte causes a change in the proximity of the electro-active moiety with respect to a surface e.g., of the respective electrode. The proximity change resulting from the aptamer interacting with, for example binding, the analyte could, for instance, result in the electro-active moiety moving closer or further from to the surface than when the aptamer is not interacting with the analyte.

The capture species are configured to bind to an epitope comprising amino acids within the particular disclosed sequence. Within means that this the epitope may comprise some or all of the amino acids within the recited sequence. It will be appreciated that the capture species in question may bind to more than just these amino acids. It will also be appreciated that this applies equally to any epitope disclosed herein, for example in the context of detection species.

In some embodiments, each surface or capture region is functionalized with the capture species. Such functionalization can be achieved in any suitable manner, such as by covalently or non-covalently immobilizing the capture species to the surface. For example, thiol-terminated capture species, such as a thiol-terminated aptamer, can be immobilized, for example grafted, onto the surface of a noble metal, for example gold or platinum, electrode.

Similarly, the first, second and third detection species, where present, make in some embodiments may comprise first, second and third detection antibodies or aptamers. The detection antibodies or aptamers may be provided with a label or tag that is detectable. Suitable labels or tags are selected based on the desired mode of detection, e.g., electrochemical, colorimetric, or optical detection.

### System for Signal Processing

In certain embodiments, a system for determining a concentration of proBNP, BNP and NT-proBNP in a sample comprises a device (e.g., flow device) according to any of the embodiments and examples comprising electrode sets described herein, and a signal processing unit configured to process signals received from the electrode sets. The system further includes a concentration determination unit configured to, based on the signals processed from the sets of electrodes and on the signals processed, determine the concentration of proBNP, BNP and NT-proBNP in the sample matrix.

The signal processing unit and the concentration determination unit may be implemented in any suitable manner, with software and/or hardware, to perform the various functions required. One or both of the units may, for example, employ one or more microprocessors programmed using software (for example, microcode) to perform the required functions. Examples of processor components that may be employed in various embodiments of the present disclosure include, but are not limited to, conventional microprocessors, application specific integrated circuits (ASICs), and field-programmable gate arrays (FPGAs). In various implementations, one or both of the signal processing unit and the concentration determination unit may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into the signal processing unit and/or the concentration determination unit.

In some non-limiting examples, the system includes a user interface, such as a display, for communicating the analyte concentration determined by the concentration determination unit.

Alternatively or additionally, the system may include a communications interface device, such as a wireless transmitter, configured to transmit the analyte concentration determined by the concentration determination unit to an external device, such as a personal computer, tablet, smartphone, remote server, etc.

### Exemplary Detection Systems

The present disclosure is at least in part based on the discovery that particular arrangements of the capture regions for proBNP, BNP and NT-proBNP can improve the differentiation between these natriuretic peptides, thereby providing assays for the diagnosis of CHF that are more accurate. In particular, the capture regions are arranged in a manner that minimizes cross-reactivity between the peptides and avoids assay pollution by proBNP.

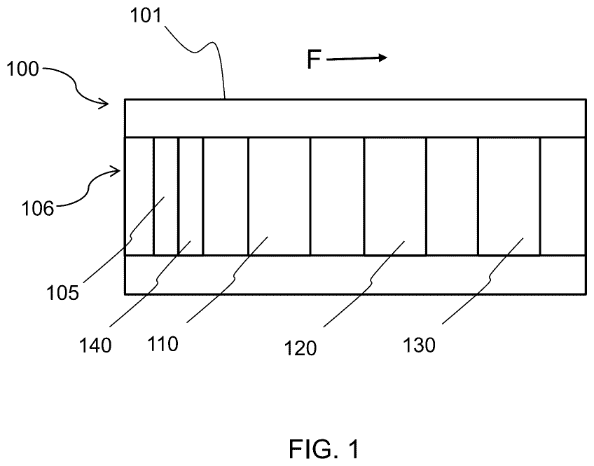

FIG. 1 provides a schematic plan view of a lateral flow assay assembly 100 for capturing and detecting the quantity of proBNP, BNP and NT-proBNP in a sample according to an embodiment. The lateral flow assay assembly 100 comprises a substrate 101 on which is provided a flow channel 106 which extends from a first end to a second end in flow direction F. A sample receiving region 105, which may be a sample pad, is located at the first end of the flow channel 106 and a reservoir region 140 is provided downstream of the sample receiving region. In the flow direction F is then provided a first capture region 110, a second capture region 120 and a third capture region 130.

The reservoir region 140, which may be a conjugate release pad, comprises a first detection antibody, a second detection antibody and a third detection antibody. The first and third detection antibodies each bind to a distinct epitope on proBNP as defined by SEQ ID NO:2. The distinct epitopes bound by the first and third detection antibodies are different from each of the epitopes bound by the first, second and third capture antibody. The capture antibodies are provided with a detectable tag (e.g., a colored tag) so that each analyte, in this case proBNP, BNP and NT-proBNP, is detectable within each of the capture regions 110, 120, 130.

The first capture region comprises a first capture antibody which binds to an epitope comprising amino acids 72-85 of SEQ ID NO: 2 (e.g., mAb Hinge76). The first capture antibody specifically binds to proBNP such that proBNP in the sample will be retained in the first capture region 110. In this embodiment, there is an excess of first capture antibodies so as to retain substantially all of the proBNP in the sample. The first capture antibody (e.g., mAb Hinge76) does not bind NT-proBNP or BNP such that BNP and NT-proBNP will not be retained by the first capture region 110 and can pass on to second and third capture regions 120, 130.

The second capture region 120 comprises a second capture antibody that binds to an epitope comprising amino acids 87-97 of SEQ ID NO: 2 (e.g., mAb 24C5, or a similar antibody that binds to the stable loop region of BNP). This second capture antibody will bind to BNP in the sample, but not NT-proBNP, thus retaining BNP in the second capture region 120. In this embodiment, there is an excess of second capture antibodies so as to retain substantially all of the BNP in the sample within the second capture region 120.

The third capture region 130 comprises a third capture antibody that binds to an epitope within a region comprising amino acids 10-76 of SEQ ID NO: 2 (e.g., mAb NT34 or a similar antibody that binds to a non-terminal and non-glycosylated epitope on NT-proBNP). This third capture antibody will bind to NT-proBNP in the sample and thus will retain NT-proBNP in the third capture region 130. In this embodiment, there is an excess of third capture antibodies so as to retain substantially all of the NT-proBNP in the sample within the third capture region 130. In some embodiments, an antibody that binds to a non-terminal and non-glycosylated epitope on NT-proBNP is used to capture NT-proBNP (e.g., mAb NT34).

In use, a sample to be analyzed, such as blood, plasma, serum, or saliva, is placed in the sample receiving region 105, which may also include additional components such as a buffer and a medium. The sample flows along the flow channel 106 of the substrate 101 in the flow direction F and first reaches the reservoir region 140 where the first, second and third detection antibodies bind to the proBNP, BNP and NT-proBNP in the sample. The sample continues to flow to the first capture region 110 where proBNP in the sample is retained, followed by the second capture region 120 where BNP in the sample is retained and then the third capture region 130 where NT-proBNP in the sample is retained. Since the first capture region 110 will selectively retain proBNP in the sample, the second capture region 120 will only encounter BNP and therefore selectively bind BNP in the sample. The detectable tags provided by the first, second and third detection antibodies will provide an indication as to the quantity of each of the analytes captured in the respective first, second and third capture regions 110, 120, 130 and therefore provide an indication of the amounts, and relative amounts, of each of the analytes (i.e., proBNP, BNP, and NT-proBNP).

Although in the above embodiment the second capture region 120 is located upstream of the third capture region 130, it will be appreciated that embodiments may have this arrangement in reverse. That is, the third capture region 130 may be located before the second capture region 120 in the flow direction (i.e., upstream).

In the above embodiments, detection is as a result of detection antibodies which are provided with a tag or label which can be detected. The tag or label may be a label which is visible without further equipment (such as a colorant) or may require an additional detection apparatus, such as a fluorescent light source.

In a further modification of this embodiment, the first capture region 110 may comprise a first capture antibody which binds to a conformational epitope comprising amino acids within (i) a first region defined by amino acids 72-85 of SEQ ID NO: 2 and (ii) a second region defined by amino acids 98-105 of SEQ ID NO: 2. This first capture antibody accordingly specifically binds to proBNP, but does not bind NT-proBNP or BNP.

In some embodiments, the first capture region comprises mAb Hinge76 as the first capture antibody, the second capture region comprises mAb 24C5 as the second capture antibody, and the third capture region comprises mAb NT34 as the third capture antibody. In this embodiment, the first detection antibody and the second detection antibody can be the same antibody (e.g., 50E1 or BC203). Alternatively, the first detection antibody and the third detection antibody can be the same (e.g., 15C4).

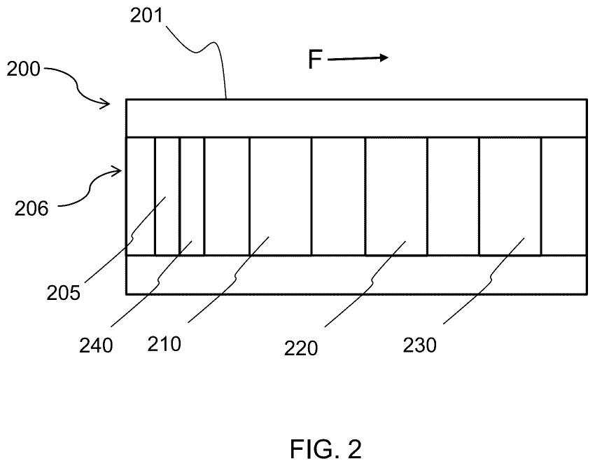

FIG. 2 provides a schematic plan view of a flow device 200 for detecting proBNP, BNP and NT-proBNP in a sample according to an embodiment. The flow device 200 comprises a substrate 201 on which is provided a flow channel 206 which extends from a first end to a second end in flow direction F. A sample receiving region 205, which may be a sample pad, is located at the first end of the flow channel 206 and a reservoir region 240 is provided downstream of the sample receiving region 202. In the flow direction F is then provided a first capture region 210, a second capture region 220 and a third capture region 230.

The reservoir region 240, which may be a conjugate release pad, comprises a first detection antibody. The first antibody binds to an epitope on proBNP as defined by SEQ ID NO:2, namely it binds to an epitope within a region comprising amino acids 10-76 of SEQ ID NO: 2 (e.g., 16F3, 29D12, 13G12 and NT34). The first detection antibody is provided with a detectable tag (e.g., a colored tag) so that analytes comprising the epitope to which it binds (i.e., proBNP and NT-proBNP) is detectable to provide a first detection signal.

The first capture region 210 comprises a first capture antibody that binds to an epitope within a region comprising amino acids 77-108 of SEQ ID NO: 2 (e.g., mAbs 50E1 or and BC203). The first capture antibody specifically binds to the BNP segment, such that proBNP and BNP in the sample will be retained in the first capture region 210.

The second capture region 220 comprises a second capture antibody that binds to an epitope comprising amino acids 87-97 of SEQ ID NO: 2 (e.g., mAb 24C5 or a similar antibody that binds to the stable loop region of BNP). This second capture antibody will bind to any unbound BNP remaining in the sample, but not NT-proBNP, thus retaining BNP in the second capture region 220.

The third capture region 230 comprises a third capture antibody that binds to an epitope within a region comprising amino acids 10-76 of SEQ ID NO: 2 (e.g., mAb NT34, or a similar antibody that binds to a non-terminal and non-glycosylated epitope on NT-proBNP), wherein the epitope bound by the first detection antibody is different from the epitope bound by the third capture antibody. This third capture antibody will bind to NT-proBNP in the sample and thus will retain NT-proBNP in the third capture region 230. This will provide an accurate concentration of NT-proBNP without any pollution from proBNP.

According, the flow device 200 of this embodiment differs from that of the lateral flow assay assembly 100 depicted in FIG. 1 in the way in which BNP and proBNP are differentiated. In particular, the flow device 200 in the present embodiment depicted in FIG. 2 uses a first capture antibody can capture both proBNP and BNP, but the detection is done in a manner that distinguishes proBNP from BNP. The flow device 200 of this embodiment is configured such that binding of BNP and proBNP in the first capture region produces a second detectable signal, where the second signal is distinguishable from the first signal.

Specifically, the flow device 200 is configured such that a second signal is produced indicative of the amount of BNP and proBNP bound in the first capture region 210. The segment labelled with the first detection antibody and associated label (i.e., the region comprising amino acids 10-76 of SEQ ID NO: 2) can be detected so as to provide a second signal corresponding to proBNP and distinguishing BNP which does not comprise this region and thus the labelled detection antibodies. These signals can be compared to provide an indication as to the quantity and presence of each of proBNP and BNP within the first capture region 210.

For example, in one specific embodiment, the first capture region 210 may comprise an electrode set with electrode(s) functionalized with the first capture antibody. The presence of BNP and proBNP bound to the electrode set can be detected by the change in potential. The label of the first detection antibody may produce a different signal, and may for example be a fluorescent label. Each signal can be quantified to determine the quantity of proBNP and BNP together (second signal provided by the electrode) and of BNP only (signal corresponding to the fluorescent label).

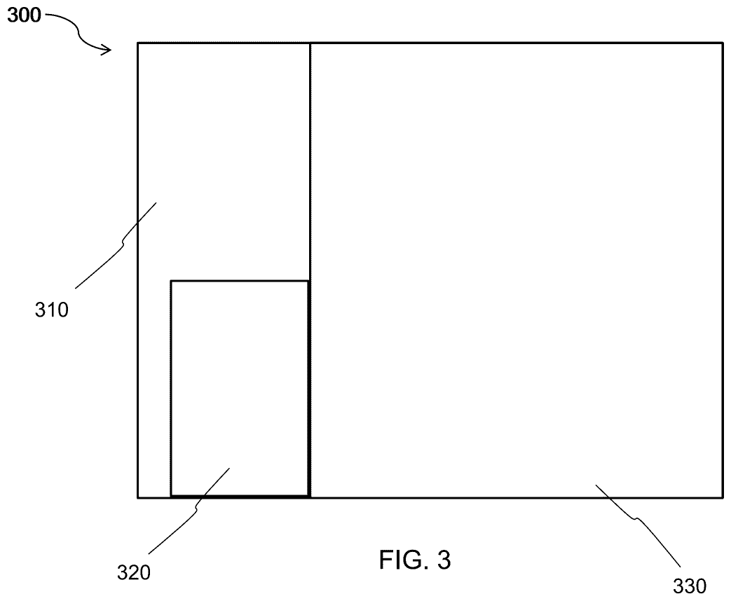

FIG. 3 shows an embodiment of a device 300 for detecting proBNP, BNP and NT-proBNP in a sample according to an embodiment. The device 300 comprises a first capture region 310 comprising a first electrode set, a second capture region 320 comprising a second electrode set and a third capture region 330 comprising a third electrode set.

Each electrode set in the first second and third capture regions is functionalized with a particular capture antibody. That is, each region comprises a set of electrodes, with at least one electrode in each set. Each electrode in the set comprises a metal surface on which is bound a capture antibody. The first capture region 310 comprises a first capture antibody which binds to a conformational epitope comprising amino acids within (i) a first region defined by amino acids 72-85 of SEQ ID NO: 2 and (ii) a second region defined by amino acids 98-105 of SEQ ID NO: 2. The first capture antibody specifically binds to proBNP. The second capture region 320 comprises a second capture antibody that binds to an epitope comprising amino acids 87-97 of SEQ ID NO: 2 (e.g., mAb 24C5 or a similar antibody that binds to the stable loop region of BNP). This second capture antibody will bind to BNP in the sample. The third capture region 330 comprises a third capture antibody that binds to an epitope within a region comprising amino acids 10-76 of SEQ ID NO: 2 (e.g., mAb NT34, or a similar antibody that binds to a non-terminal and non-glycosylated epitope on NT-proBNP). This third capture antibody will bind to NT-proBNP in the sample.

In this embodiment, the density/surface area of the capture antibodies on the electrode sets (and thus the capture regions 310, 320, 330) are provided in a ratio corresponding to the expected ratio of the proBNP:BNP:NT-proBNP in the sample. Specifically, the expected ratio of proBNP:BNP is roughly 1.5:1. The amount of NT-proBNP expected is roughly 6 times BNP such that the capture areas in this embodiment are provided in a ratio of 1.5:1:6 proBNP:BNP:NT-proBNP.

Each electrode set is connected to a measurement system which can address each set of electrodes to determine a response which is dependent on whether samples are bound to the capture molecules and output a signal. The potential of the electrode changes based on the binding of the target analyte to the capture antibody such that the signal will change. Reference and counter electrodes are provided (not shown).

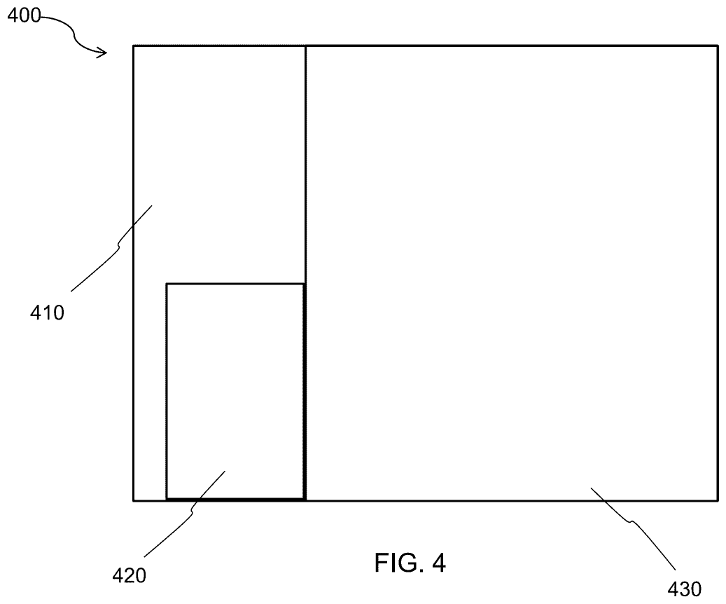

FIG. 4 shows an embodiment of a device 400 for detecting proBNP, BNP and NT-proBNP in a sample according to an embodiment. The device 400 comprises a first capture region 410 comprising a first electrode set, a second capture region 420 comprising a second electrode set and a third capture region 430 comprising a third electrode set. The capture regions 410, 420, 430 are arranged in the shape and configuration (with electrodes functionalized with the respective capture antibody), with the only exception being that the first capture antibody in the first capture region 410 in this embodiment is a first capture antibody which binds to an epitope comprising amino acids 72-85 of SEQ ID NO: 2. Accordingly, in this embodiment, the binding of proBNP and BNP can be achieved by the first capture antibody.

Selective binding in this case will be controlled by two factors: (i) diffusion-based binding and (ii) relative number of antibodies. The relative number of capture antibodies available for each peptide sequence can control selective binding through diffusion-based binding. Moreover, when sample is added to the device, lighter molecules travel faster and bind appropriate antibodies before heavier molecules reach that capture region. The capture regions are already sized to the expected proportion of target molecules expected (in the ratio set out for FIG. 3). Accordingly, the geographical separation of capture antibodies will ensure that the corresponding measurement system will record the appropriate peptide signals.

In further configurations, the systems may be further manipulated to determine the amount and size of the bound analytes. For example, manipulation of the capture antibodies under the effect of an electrical field may cause movement of the antibodies relative to the surface, altering the signal over time. The speed of movement of a part of the capture antibody towards and away from the surface is altered by the weight of the bound analyte such that analytes of different weight (or even of the absence of any analyte) can be used to further distinguish or quantify binding.

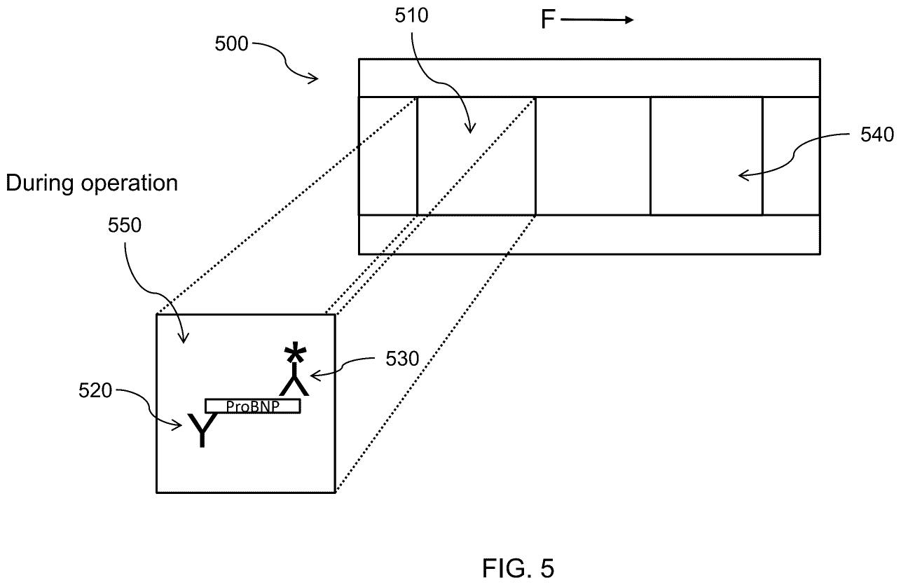

FIG. 5 shows a device 500 for detecting proBNP in a sample. The device 500 comprises a first capture region 510 comprising a first capture antibody 520 that binds to an epitope within a region comprising amino acids 1-76 of SEQ ID NO: 2 (e.g., an epitope within a region comprising amino acids 10-76 of SEQ ID NO: 2, such as an epitope comprising amino acids 27-31 of SEQ ID NO: 2, e.g., mAb NT34). The detection device 500 additionally comprises a second capture region 540 comprising a second capture antibody. The second capture antibody may bind to an epitope comprising amino acids 87-93 of SEQ ID NO: 2 (e.g., mAb 24C5 or a similar antibody that binds to the stable loop region of BNP). The second capture region 540 is located downstream of the first capture region 510 (the flow direction F is indicated by an arrow). BNP comprised in the sample is not bound by the first capture region 510 and thus is able to bind to the second capture antibody in the second capture region 540.

When in operation, a sample (e.g., blood, plasma, serum, or saliva) is added to the first capture region of the device 500, and proBNP comprised within the sample is bound by the first capture antibody 520. After addition of the sample, the first capture region 510 may be washed to remove any unbound material. Next, a solution comprising a first detection antibody 530 comprising a detectable label (indicated by an asterisk) is added to the first capture region 510. In a typical embodiment, the detectable label is a fluorescent moiety.

In some embodiments, the first detection antibody 530 binds to an epitope comprising amino acids 72-85 of SEQ ID NO: 2 (e.g., mAb Hinge76). In this embodiment, the first detection antibody specifically binds to proBNP and does not bind to NT-proBNP or BNP. Accordingly, a second detection antibody comprising a detectable label is required for the detection of BNP. In a typical embodiment, the second detection antibody binds to an epitope comprising amino acids 87-93 of SEQ ID NO: 2 (or to the complex of BNP and the capture antibody, e.g., Ab-BNP2). Accordingly, the solution comprising the first detection antibody 530 may also comprise the second detection antibody.

In other embodiments, the first detection antibody 530 binds to an epitope comprising amino acids 87-93 of SEQ ID NO: 2. In certain embodiments, a single detection antibody may be used to detect proBNP in the first capture region 510 and BNP in the second capture region 540 (e.g., 50E1 or BC203).

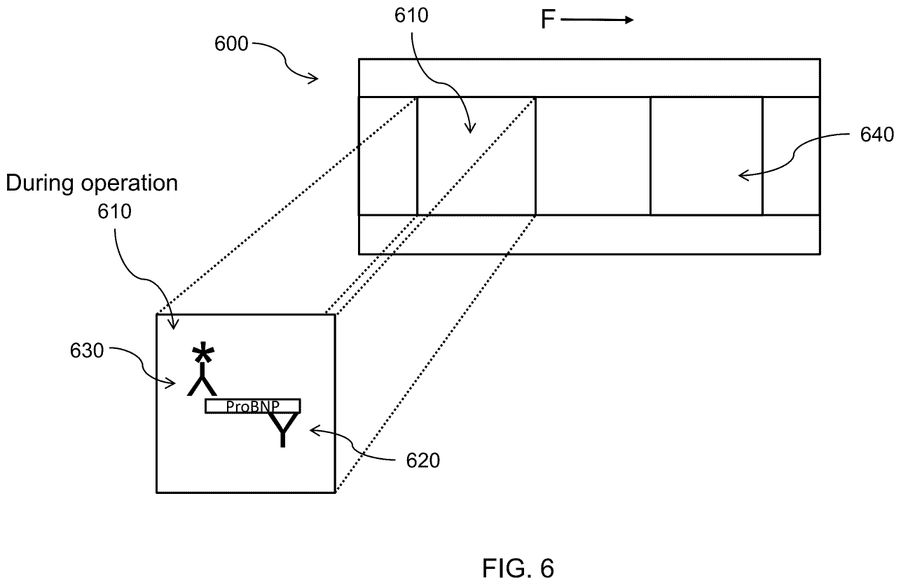

FIG. 6 shows a device 600 for detecting proBNP in a sample. The device 600 comprises a first capture region 610 comprising a first capture antibody 620. In some embodiments, the first capture antibody binds to an epitope comprising amino acids 72-85 of SEQ ID NO: 2 including the site at which proBNP is cleaved into NT-proBNP and BNP (e.g., mAb Hinge76). In other embodiments, the first capture antibody 620 binds to a conformational epitope comprising amino acids within (i) a first region defined by amino acids 72-85 of SEQ ID NO: 2 and (ii) a second region defined by amino acids 98-105 of SEQ ID NO: 2. Typically, in either embodiment, the first capture antibody specifically binds to proBNP and does not bind NT-proBNP or BNP. The detection device 600 additionally comprises a second capture region 640 comprising a second capture antibody that binds to an epitope comprising amino acids 87-93 of SEQ ID NO: 2 (e.g., mAb 24C5 or a similar antibody that binds to the stable loop region of BNP). The second capture region 640 is located downstream of the first capture region 610 (the flow direction F is indicated by an arrow). BNP comprised in the sample is not bound by the first capture region 610 and thus is able to bind to the second capture antibody in the second capture region 640.

When in operation, a sample is added to the first capture region of the device 600, and proBNP comprised within the sample is bound by the first capture antibody 620. After addition of the sample, the first capture region 610 may be washed to remove any unbound material. Next, a solution comprising a first detection antibody 630 comprising a detectable label (indicated by an asterisk) is added to the first capture region 610. In a typical embodiment, the detectable label is a fluorescent moiety.

In some embodiments, the first detection antibody 630 binds to an epitope within a region comprising amino acids 1-76 of SEQ ID NO: 2 (e.g., an epitope within a region comprising amino acids 10-76 of SEQ ID NO: 2 such as an epitope comprising amino acids 27-31 of SEQ ID NO: 2, e.g., NT34 mAb). In this embodiment, the first detection antibody binds to proBNP as well as NT-proBNP. A second detection antibody comprising a detectable label is required for the detection of BNP. In a typical embodiment, the second detection antibody binds to a complex formed by the second capture antibody and BNP (e.g., Ab-BNP2). Accordingly, the solution comprising the first detection antibody 630 may also comprise the second detection antibody.

Although the exemplary detection systems described above refer to the use of antibodies as capture and detection species, alternative capture and detection species (e.g., aptamers) may be used in the implementation of the present disclosure.

### Select Examples

Example 1 provides a device for determining the presence of BNP, proBNP and NT-proBNP in a sample, the device including: a first capture region including a first capture species, wherein the first capture species binds to an epitope that is located within a region defined by amino acids 72-105 of SEQ ID NO: 2, wherein the epitope either includes amino acids 75-78 of SEQ ID NO: 2, or includes amino acids located on either side of a cleavage site between amino acid 76 and amino acid 77 of SEQ ID NO: 2, wherein said first capture species specifically binds to proBNP, but does not bind NT-proBNP or BNP; a second capture region including a second capture species that binds to an epitope including amino acids within 87-97 of SEQ ID NO: 2; and a third capture region including a third capture species that binds to an epitope within a region including amino acids 10-76 of SEQ ID NO: 2.

Example 2 provides a device for determining the presence of BNP, proBNP and NT-proBNP in a sample, the device including: a first capture region including a first capture species, wherein the first capture species binds to an epitope including amino acids 72-85 of SEQ ID NO: 2, wherein said first capture species specifically binds to proBNP, but does not bind NT-proBNP or BNP; a second capture region including a second capture species that binds to an epitope including amino acids within 87-97 of SEQ ID NO: 2; and a third capture region including a third capture species that binds to an epitope within a region including amino acids 10-76 of SEQ ID NO: 2.

Example 3 provides a device for determining the presence of BNP, proBNP and NT-proBNP in a sample, the device including: a first capture region including a first capture species binds to a conformational epitope including amino acids within (a) a first region defined by amino acids 72-85 of SEQ ID NO: 2 and (b) a second region defined by amino acids 98-105 of SEQ ID NO: 2; wherein said first capture species specifically binds to proBNP, but does not bind NT-proBNP or BNP; a second capture region including a second species that binds to an epitope including amino acids within 87-97 of SEQ ID NO: 2; and a third capture region including a third capture species that binds to an epitope within a region including amino acids 10-76 of SEQ ID NO: 2.

Example 4 provides a device for determining the presence of BNP, proBNP and NT-proBNP in a sample, the device including: a reservoir region including a first detection species, wherein the first detection species includes a first label detectable to produce a first signal and binds to either (i) a first epitope within a region including amino acids 10-76 of SEQ ID NO: 2 or (ii) a second epitope within a region including amino acids 77-108 of SEQ ID NO: 2; a first capture region including a first capture species that binds to the other of (i) a first epitope within a region including amino acids 10-76 of SEQ ID NO: 2 or (ii) a second epitope within a region including amino acids 77-108 of SEQ ID NO: 2, such that one of the first capture species and the first detection species binds to the first epitope and the other of the first capture species and the first detection species binds to the second epitope; a second capture region including a second capture species, wherein the second capture species binds to a third epitope located within the same region as the epitope bound by the first detection species; and optionally a third capture region including a third capture species, wherein the third capture species binds to a fourth epitope located within the same region as the epitope bound by the first capture species, wherein the device is configured such that binding of BNP and proBNP in the first capture region produces a second signal, which second signal is distinguishable from the first signal.

Example 5 provides the device according to example 4, wherein the first detection species is a first detection antibody.

Example 6 provides the device according to example 1, 2 or 3, further including a sample-receiving region; and a reservoir region including a first detection species, a second detection species and a third detection species, wherein the first and third detection species each bind to a distinct epitope on proBNP as defined by SEQ ID NO: 2, wherein the distinct epitopes bound by the first and third detection species are different from each of the epitopes bound by the first, second and third capture species.

Example 7 provides the device according to any one of the preceding examples, wherein the device is a flow device and the first capture region is located upstream of the second and third capture regions in the flow direction.

Example 8 provides the device according to any one of the preceding examples, wherein the second capture species binds to an epitope including amino acids 87-93 of SEQ ID NO: 2.

Example 9 provides the device according to any one of the preceding examples, wherein the first, second and third capture species are first, second and third capture antibodies, respectively.

Example 10 provides the device according to examples 1-3 or 6, wherein the first capture species is a first capture antibody, e.g., mAb Hinge76.

Example 11 provides the device according to any one of the preceding examples, wherein the second capture species is a second capture antibody, e.g., mAb 24C5.

Example 12 provides the device according to example 11, wherein the second detection species is a detection antibody which binds a complex formed by the second capture antibody and BNP.

Example 13 provides the device according to example 12, wherein the second detection antibody is Ab-BNP2.

Example 14 provides the device according to any one of examples 1-7, wherein the second capture species binds to an epitope including amino acids 90-97 of SEQ ID NO: 2.

Example 15 provides the device according to any one of the preceding examples, wherein the epitope bound by the third capture species is free of glycosylated amino acids.

Example 16 provides the device according to any one of the preceding examples, wherein the third capture species binds to an epitope including amino acids 27-31 of SEQ ID NO: 2.

Example 17 provides the device according to example 16, wherein the third capture species is a third capture antibody, e.g., mAb NT34.

Example 18 provides the device according to any one of the preceding examples, wherein the third detection species binds to an epitope including amino acids 42-46 of SEQ ID NO: 2.

Example 19 provides the device according to any one of examples 1-15, wherein the third capture species binds to an epitope including amino acids 63-71 of SEQ ID NO: 2.

Example 20 provides the device according to example 19, wherein the third capture species is a third capture antibody, e.g., mAb 15C4.

Example 21 provides the device according to example 19 or 20, wherein the third detection species binds to an epitope including amino acids 13-20 of SEQ ID NO: 2.

Example 22 provides the device according to example 21, wherein the third detection species is a third detection antibody, e.g., mAb 13G12.

Example 23 provides the device according to any one of the preceding examples, wherein the first, second and third capture regions are distinct segments on a nitrocellulose membrane.

Example 24 provides the device according to any one of the preceding examples, wherein the first capture region includes a first electrode set including at least one electrode, wherein the first capture species is bound to a surface of the electrode of the first electrode set; wherein the second capture region includes a second electrode set including at least one electrode, wherein the second capture species is bound to a surface of the electrode of the second electrode set; wherein the third capture region includes a third electrode set including at least one electrode, wherein the third capture species is bound to a surface of the electrode of the third electrode set.

Example 25 provides the device according to any one of the preceding examples, wherein the flow device is capable of separating proBNP, BNP and NT-proBNP by size.

Example 26 provides the device according to any one of the preceding examples, wherein the first, second and third capture species in the first, second and third capture regions are present at a ratio of about 1.5:1:6.

Example 27 provides a method for monitoring a subject suffering from heart disease, said method including: applying a sample obtained from the subject to sample-receiving region of the flow device according to any one of examples 1-26; and detecting the presence of NT-proBNP and BNP in the sample.

Example 28 provides the method of example 27, wherein detecting the presence of NT-proBNP and BNP in the sample indicates that the subject is suffering from heart failure with reduced ejection fraction.

Example 29 provides the method of example 27 or 28, wherein the subject is receiving Angiotensin Receptor and Neprilysin Inhibitor (ARNI) therapy.

Example 30 provides a kit including the device according to any one of examples 1-22 and instructions for use in the method of any one of examples 27-29.

It should be understood that the detailed description and specific examples, while indicating exemplary embodiments of the apparatus, systems and methods, are intended for purposes of illustration only and are not intended to limit the scope. These and other features, aspects, and advantages of the apparatus, systems and methods of the present disclosure can be better understood from the description, appended claims or aspects, and accompanying drawings. It should be understood that the Figures are merely schematic and are not drawn to scale. It should also be understood that the same reference numerals are used throughout the figures to indicate the same or similar parts.

Other variations to the disclosed embodiments can be understood and effected by those skilled in the art in practicing the disclosure, from a study of the drawings, the disclosure, and the appended aspects or claims. In the aspects or claims, the word “comprising” does not exclude other elements or steps, and the indefinite article “a” or “an” does not exclude a plurality. The mere fact that certain measures are recited in mutually different dependent aspects or claims does not indicate that a combination of these measures cannot be used to advantage. Any reference signs in the claims should not be construed as limiting the scope.

What is claimed is:

1. A device for determining the presence of BNP, proBNP and NT-proBNP in a sample, the device comprising:
(i) a first capture region comprising a first capture species, wherein the first capture species binds to an epitope that is located within a region defined by amino acids 72-105 of SEQ ID NO: 2, wherein the epitope either comprises amino acids 75-78 of SEQ ID NO: 2, or comprises amino acids located on either side of a cleavage site between amino acid 76 and amino acid 77 of SEQ ID NO: 2, wherein said first capture species specifically binds to proBNP, but does not bind NT-proBNP or BNP;
(ii) a second capture region comprising a second capture species that binds to an epitope comprising amino acids within 87-97 of SEQ ID NO: 2; and
(iii) a third capture region comprising a third capture species that binds to an epitope within a region comprising amino acids 10-76 of SEQ ID NO: 2.

2. The device according to claim 1, further comprising:
a sample-receiving region; and
a reservoir region comprising a first detection species, a second detection species and a third detection species, wherein the first and third detection species each bind to a distinct epitope on proBNP as defined by SEQ ID NO: 2,
wherein the distinct epitopes bound by the first and third detection species are different from each of the epitopes bound by the first, second and third capture species.

3. A device for determining the presence of BNP, proBNP and NT-proBNP in a sample, the device comprising:
(i) a first capture region comprising a first capture species, wherein the first capture species binds to an epitope comprising amino acids 72-85 of SEQ ID NO: 2, wherein said first capture species specifically binds to proBNP, but does not bind NT-proBNP or BNP;
(ii) a second capture region comprising a second capture species that binds to an epitope comprising amino acids within 87-97 of SEQ ID NO: 2; and
(iii) a third capture region comprising a third capture species that binds to an epitope within a region comprising amino acids 10-76 of SEQ ID NO: 2.

4. The device according to claim 3, further comprising:
a sample-receiving region; and
a reservoir region comprising a first detection species, a second detection species and a third detection species, wherein the first and third detection species each bind to a distinct epitope on proBNP as defined by SEQ ID NO: 2,
wherein the distinct epitopes bound by the first and third detection species are different from each of the epitopes bound by the first, second and third capture species.

5. A device for determining the presence of BNP, proBNP and NT-proBNP in a sample, the device comprising:
(i) a reservoir region comprising a first detection species, wherein the first detection species comprises a first label detectable to produce a first signal and binds to either (i) a first epitope within a region comprising amino acids 10-76 of SEQ ID NO: 2 or (ii) a second epitope within a region comprising amino acids 77-108 of SEQ ID NO: 2;
(ii) a first capture region comprising a first capture species that binds to the other of (i) a first epitope within a region comprising amino acids 10-76 of SEQ ID NO: 2 or (ii) a second epitope within a region comprising amino acids 77-108 of SEQ ID NO: 2, such that one of the first capture species and the first detection species binds to the first epitope and the other of the first capture species and the first detection species binds to the second epitope;
(iii) a second capture region comprising a second capture species, wherein the second capture species binds to a third epitope located within the same region as the epitope bound by the first detection species; and
(iv) optionally a third capture region comprising a third capture species, wherein the third capture species binds to a fourth epitope located within the same region as the epitope bound by the first capture species,
wherein the device is configured such that binding of BNP and proBNP in the first capture region produces a second signal, which second signal is distinguishable from the first signal.

6. The device according to claim 2, wherein the second detection species is a detection antibody which binds a complex formed by the second capture antibody and BNP.

7. The device according to claim 4, wherein the second capture species is a second capture antibody and wherein the second detection species is a detection antibody which binds a complex formed by the second capture antibody and BNP.

8. The device according to claim 5, wherein the second capture species is a second capture antibody and wherein the second detection species is a detection antibody which binds a complex formed by the second capture antibody and BNP.

9. The device according to claim 2, wherein the third detection species binds to an epitope comprising amino acids 42-46 of SEQ ID NO: 2 or amino acids 13-20 of SEQ ID NO: 2.

10. The device according to claim 4, wherein the third detection species binds to an epitope comprising amino acids 42-46 of SEQ ID NO: 2 or amino acids 13-20 of SEQ ID NO: 2.

11. The device according to claim 5, wherein the third detection species binds to an epitope comprising amino acids 42-46 of SEQ ID NO: 2 or amino acids 13-20 of SEQ ID NO: 2.

12. The device according to claim 1, wherein the first, second and third capture species in the first, second and third capture regions are present at a ratio of about 1.5:1:6.

13. The device according to claim 3, wherein the first, second and third capture species in the first, second and third capture regions are present at a ratio of about 1.5:1:6.

14. The device according to claim 5, wherein the first, second and third capture species in the first, second and third capture regions are present at a ratio of about 1.5:1:6.

15. A method for monitoring a subject suffering from heart disease, said method comprising:
(i) applying a sample obtained from the subject to sample-receiving region of the flow device according to any one of claims 1, 3 or 5;
(ii) detecting the presence of NT-proBNP and BNP in the sample.

16. The method of claim 15, wherein detecting the presence of NT-proBNP and BNP in the sample indicates that the subject is suffering from heart failure with reduced ejection fraction.
